(19) United States
(12) Patent Application Publication
(10) Pub. No.: US 2013/0103137 A1
(43) Pub. Date: Apr. 25, 2013

(54) ARTIFICIAL BLOOD VESSEL

(71) Applicants: Takuji Asano, Shibuya-ku (JP); Daisuke Yokoyama, Shibuya-ku (JP); Yoshihiko Kinoshita, Shibuya-ku (JP)

(72) Inventors: Takuji Asano, Shibuya-ku (JP); Daisuke Yokoyama, Shibuya-ku (JP); Yoshihiko Kinoshita, Shibuya-ku (JP)

(73) Assignee: NIKKISO CO., LTD., Tokyo (JP)

(21) Appl. No.: 13/643,131

(22) PCT Filed: Jun. 13, 2011
(86) PCT No.: PCT/JP2011/063481

§ 371 Date: Oct. 24, 2012

(30) Foreign Application Priority Data
Jul. 7, 2010 (JP) 2010-154749
May 27, 2011 (JP) 2011-118952

(51) Int. Cl. A61F 2/856

(52) U.S. Cl. CPC A61F 2/856
USPC 623/1.35

(57) ABSTRACT

An artificial blood vessel has a trunk portion which forms a part of the blood vessel and also has branch portions which are branched from the trunk portion. The branch portions are provided with access ports into which a needle, for discharging liquid out of a human body or introducing the liquid into the human body, is inserted. Tubular reinforcement members integrated with the blood vessel wall of the trunk portion are disposed at positions of the trunk portion, the positions being those from which the branch portions are branched, and at portions adjacent to the positions of the trunk portion. A lateral force at the time of needle insertion is borne by the reinforcement member, and this prevents the trunk portion from deforming. Thus, the configuration prevents the artificial blood vessel from deforming due to a force applied when a needle for discharging liquid from a human body or introducing the liquid into the human body is inserted into an access port of the artificial blood vessel.

### TECHNICAL FIELD

The present invention relates to an artificial blood vessel, and in particular, to a structure of an artificial blood vessel.

### BACKGROUND ART

In extracorporeal blood circulation treatments in which blood of a patient is temporarily extracted from the body and then returned into the body, such as hemodialysis and apheresis treatment, it is necessary to highly frequently insert a needle into the blood vessel. When the needle is highly-frequently inserted into the blood vessel, there are cases where an aneurysm may be formed or vasoconstriction may be caused. A blood access (blood vessel reaching method) has been proposed which can reduce the number of needle insertions into the blood vessel and which uses an indwelling device. Patent Document 1 discloses a blood access in which a blood chamber connected with the blood vessel by a cannula is equipped with a diaphragm, a needle is inserted into the diaphragm, and introduction of blood or the like into the blood vessel and extraction of blood from the blood vessel or the like are performed through the needle. Because the needle is repeatedly inserted into the diaphragm, damage to the blood vessel can be reduced.

Patent Literature 2 discloses an example structure where an artificial blood vessel has a stent for maintaining the shape of the artificial blood vessel.

### RELATED ART REFERENCES

### Patent Literature

[Patent Literature 1] JP H9-510885 A
[Patent Literature 2] JP 2005-58434 A

### DISCLOSURE OF INVENTION

### Summary of the Invention

### Problem to be Solved by the Invention

When a force from a lateral direction is applied to an artificial blood vessel which is indwelled in the body, there may be cases where the artificial blood vessel is deformed and the bloodstream is blocked. The stent disclosed in Patent Literature 2 assumes intravascular treatment, and is extensible. In other words, when the stent is placed in the body, the stent is in a retracted state, and after the stent is placed, the stent is extended and is maintained in this state. Such a stent does not take into consideration a case where an external force, in particular, a lateral force, acts on the blood vessel.

An advantage of the present invention is provision of an artificial blood vessel which can resist a lateral force.

### Means for Achieving the Objects

According to one aspect of the present invention, there is provided an artificial blood vessel comprising a tubular reinforcement member integrated with a blood vessel wall, wherein the reinforcement member has rigidity to prevent closure of the artificial blood vessel when a lateral force is applied.

According to another aspect of the present invention, preferably, the artificial blood vessel comprises a trunk portion which forms apart of the blood vessel, and a branch portion branched from the trunk portion. An access port, into which a needle for extracting a fluid from the body or introducing the fluid into the body is inserted, is placed on the branch portion. The tubular reinforcement member integrated with the blood vessel wall of the trunk portion is placed at a position of the trunk portion where the branch portion branches and an adjacent portion thereof. The reinforcement member prevents closure of the artificial blood vessel when a lateral force is applied.

According to another aspect of the present invention, preferably, in the artificial blood vessel, a blood vessel wall on a portion where the reinforcement member is placed has two layers including an inner wall and an outer wall, the reinforcement member is sandwiched between the inner wall and the outer wall, and the reinforcement member is not exposed to the outside.

According to another aspect of the present invention, preferably, in the artificial blood vessel, a tubular wall of the reinforcement member has a mesh shape. According to another aspect of the present invention, preferably, in the artificial blood vessel, an opening is formed in the reinforcement member at a position where the branch portion branches, and the trunk portion and the branch portion are connected at a position of the opening. According to another aspect of the present invention, preferably, in the artificial blood vessel, the trunk portion and the branch portion are connected by suturing at the position of the opening. According to another aspect of the present invention, preferably, in the artificial blood vessel, a plurality of suture holes are arranged along a circumferential direction in a ring-shaped portion defining the opening, and the trunk portion and the branch portion are sutured by passing a suturing thread through the suture holes.

### Advantageous Effect of Invention

According to various aspects of the present invention, the reinforcement member bears the force applied from the lateral direction to the artificial blood vessel, so that the closure of the artificial blood vessel is prevented. In addition, in the artificial blood vessel having the branch portion in which the access port is placed, the lateral force applied on the trunk portion when the needle is inserted into the access port can be borne by the reinforcement member, and thus the closure of the trunk portion of the artificial blood vessel can be prevented.

### BRIEF DESCRIPTION OF DRAWINGS

FIG. 1 is a perspective diagram showing an example configuration of an artificial blood vessel having an access port.

FIG. 2 is a diagram showing a detailed shape of a reinforcement member.

FIG. 3 is a cross sectional diagram of a branching portion of an artificial blood vessel.

FIG. 4 is a cross sectional diagram of a branching portion of an artificial blood vessel.

FIG. 5 is an explanatory diagram showing insertion of a needle into an artificial blood vessel.

### BEST MODE FOR CARRYING OUT THE INVENTION

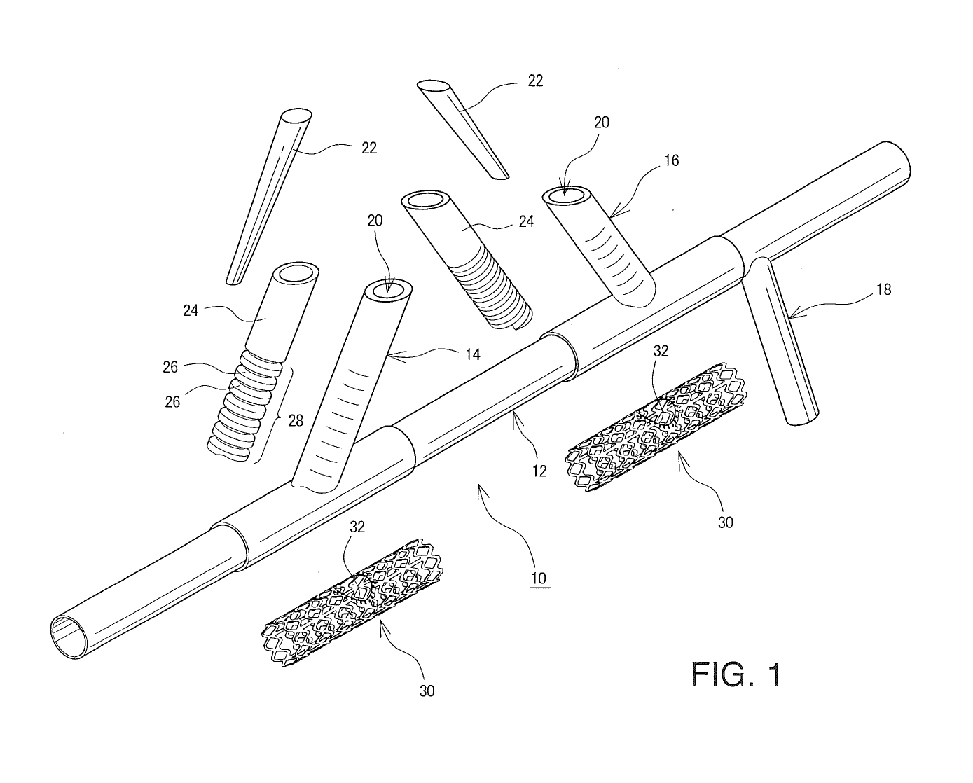

A preferred embodiment of the present invention will now be described with reference to the drawings. FIG. 1 is a perspective diagram showing an example configuration of an artificial blood vessel 10 having an access port. The artificial blood vessel 10 is used in an extracorporeal blood circulation treatment such as hemodialysis and apheresis treatment in which, after the blood is extracted to the outside of the body and a predetermined process is performed, the blood is again introduced into the body. The artificial blood vessel 10 comprises a trunk portion having both ends connected to a blood vessel, in particular, the vein, and which bridges the vein, and branch portions 14, 16, and 18 branched from the trunk portion 12. Two branch portions 14 and 16 form connection points with an extracorporeal blood circuit for extracting blood to the outside of the body or for introducing fluid such as blood into the body, and the other branch portion 18 forms a shunt to be connected to the artery. Here, the two former branch portions 14 and 16 will be called access branch portions 14 and 16 and the latter branch portion 18 will be called a shunt branch portion 18. The trunk portion 12 and the shunt branch portion 18 are indwelled in the body, and a part of the access branch portions 14 and 16 is exposed to the outside of the body. The artificial blood vessel 10 may be formed with a material for an artificial blood vessel used in the related art such as ePTFE (expanded polytetrafluoroethylene), PTFE (polytetrafluoroethylene), etc.

Portions, of the trunk portion 12, in a range of about 20 mm from the ends of the trunk portion 12 may be conjugated (coated) with SHAp (highly-dispersible nano-particles of sintered hydroxyapatite) so that the junction surface between the blood vessel of the living body and the artificial blood vessel can be smoothened, resulting in improved latency rate and, at the same time, prevention of blood leakage of initial anastomosed portion.

An access port 20 is placed in the access branch portions 14 and 16. The access port 20 has an overall hollow pillar shape, preferably, a hollow circular pillar shape, has a length equal to lengths of the branch portions 14 and 16, and fills the inside of the branch portions 14 and 16. That is, the branch portions 14 and 16 are plugged by the access port 20. The access port 20 has a center portion 22 extending along an axis of the pillar and a tubular peripheral portion 24 surrounding the periphery of the center portion. In FIG. 1, the center portion 22 and the peripheral portion 24 are separately shown. The center portion 22 is tightly fitted to the inside of the tubular shape of the peripheral portion 24, and with this configuration, the access port 20 has an overall hollow pillar shape. The tube inner wall of the tubular peripheral portion 24 may have a tapered shape narrowing toward the tip, that is, a direction toward the trunk portion 12. In correspondence to this shape, the center portion 22 also has a tapered shape narrowing toward the tip.

An angle of the above-described taper is significantly affected by the blood vessel course and a position where the port is extracted. Therefore, the taper angle is not limited to the above-described range, and may be suitably changed in design according to the anastomosed position of the artificial blood vessel and a distance to the surface skin.

The material of the access port 20 may be a resin material having elasticity, for example, a highly-compressed silicone, the center portion 22 has a hardness of a degree to allow a needle to be inserted, and for the peripheral portion, silicone having a higher hardness compared to the center portion 22 is used. The hardness of the silicone is about 10-80 degrees, but the hardness of the silicone is not limited to this range.

On an outer circumferential surface of the peripheral portion 24 on the trunk portion side, a plurality of ring-shaped grooves 26 are formed, and with this configuration, a recess-projection section 28 having a recess-projection shape is formed on the outer circumferential surface in the axial direction. With the recess-projection section 28, the recess and projection also appear on the surface of the access branch portions 14 and 16, which results in a superior matching with the living body.

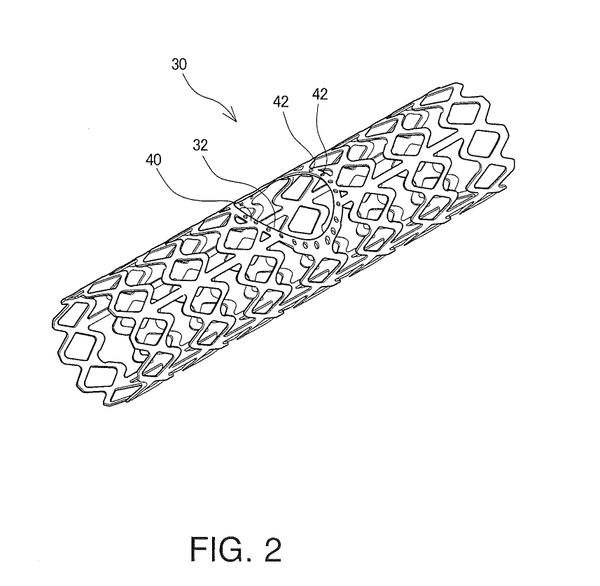

On positions of the trunk portion 12 where the branch portions 14 and 16 branch and in the portions adjacent thereto, a reinforcement member 30 for maintaining the tubular shape of the trunk portion 12 is built in. The reinforcement member 30 is sandwiched between artificial blood vessel walls formed in two layers in these portions, and is not exposed to the outside of the artificial blood vessel wall. Because of this, the reinforcement member 30 does not directly contact the blood, and thus, there is no risk of vasoconstriction. In FIG. 1, the reinforcement member 30 is independently shown, and in FIG. 2, the reinforcement member 30 is shown in an enlarged manner. The reinforcement member 30 has an approximate tubular shape and a mesh-shaped structure. As shown in the drawings, the mesh has a shape where rhombuses are combined. With the mesh structure, the recess and projection of the mesh engage the resin material of the artificial blood vessel wall, resulting in a superior matching of these structures. On a side surface at an approximate center in the length direction, openings 32 corresponding to the branch portions 14 and 16 are formed. The material of the reinforcement member 30 may be a metal such as stainless steel, nitinol (nickel-titanium alloy), etc.

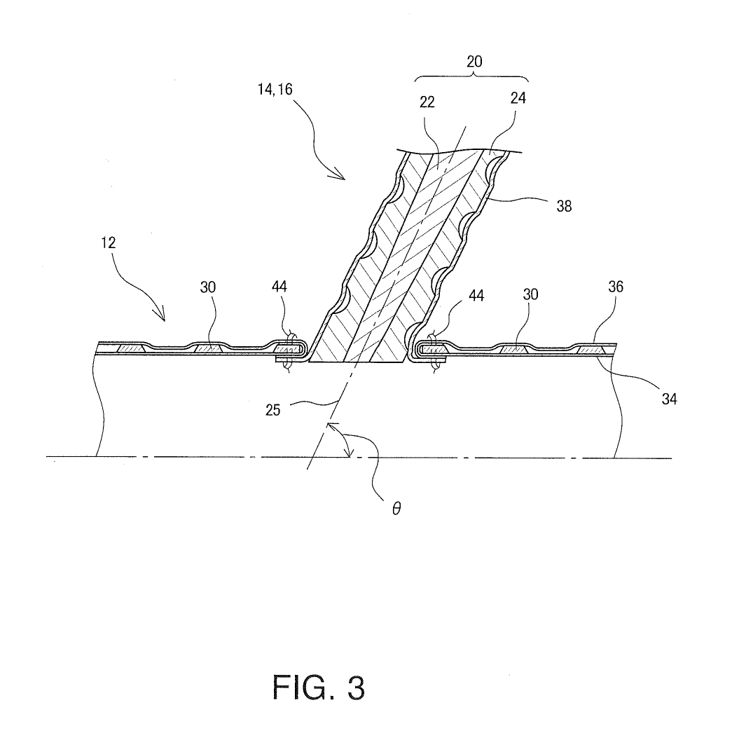

FIG. 3 is a diagram showing a cross section of positions where the trunk portion 12 and the branch portions 14 and 16 of the artificial blood vessel 10 branch, and portions adjacent to the positions. The branch position and the portion adjacent thereof will hereinafter be called a branching portion. As described before, at the branching portion, the trunk portion 12 is formed with an artificial blood vessel wall of two layers. The inner blood vessel wall will be described as an inner wall 34 and the outer blood vessel wall will be described as an outer wall 36. The reinforcement member 30 descried above is sandwiched between the inner wall 34 and the outer wall 36. The outer wall 36 is slightly longer than the reinforcement member 30 in the axial direction of the trunk portion 12 and is in close contact at both ends with the inner wall 34 over the entire periphery. Because of this configuration, the reinforcement member 30 is not exposed. In addition, at an edge of the opening 32 of the reinforcement member 30, the inner wall 34 entangles the outer side surface of the reinforcement member 30 and the outer wall 36 entangles the inner side surface of the reinforcement member 30 so that the reinforcement member 30 is not exposed at this portion either. In other words, the reinforcement member 30 is completely covered by the inner wall 34 and the outer wall 36. On the blood vessel wall 38 of the branch portions 14 and 16, recess and projection are formed on the outer surface, reflecting the recess and projection of the recess-projection section 28 formed on the outer periphery of the access port 20.

With this increase in the surface area of the blood vessel wall 38, a binding area with cells is increased, and the matching with the living body is improved. In addition, the risk of detachment between the tissue and the device when a stress in the longitudinal direction of the branch portions 14 and 16 is applied can be reduced, and the load on the junction section can be reduced.

FIG. 3 also shows a cross section of the access port 20. As shown in FIG. 3, the center portion 22 has a tapered shape narrowing toward the tip, and the peripheral portion 24 has a tapered shape for the inner wall, narrowing toward the tip. As shown in FIG. 3, the access branch portions 14 and 16 are placed in an inclined manner with an angle θ with respect to the trunk portion 12, and, corresponding to this inclined placement, a tip of the access port 20 is formed in an inclined manner with respect to the axis of the access port 20 such that the tip is on the same plane as the inner wall of the trunk portion 12. The tip of the access port 20 is formed to be on the same plane as the inner wall surface such that there is no step formed therebetween. The above-described angle θ of the inclined placement is ideally between 40 degrees and 60 degrees, but depends significantly on the blood vessel course and the position where the port is extracted. Therefore, the angle θ of the inclined placement is not limited to the above-described numerical range, and may be suitably changed in design based on the anastomosed position of the artificial blood vessel and the distance to the surface skin.

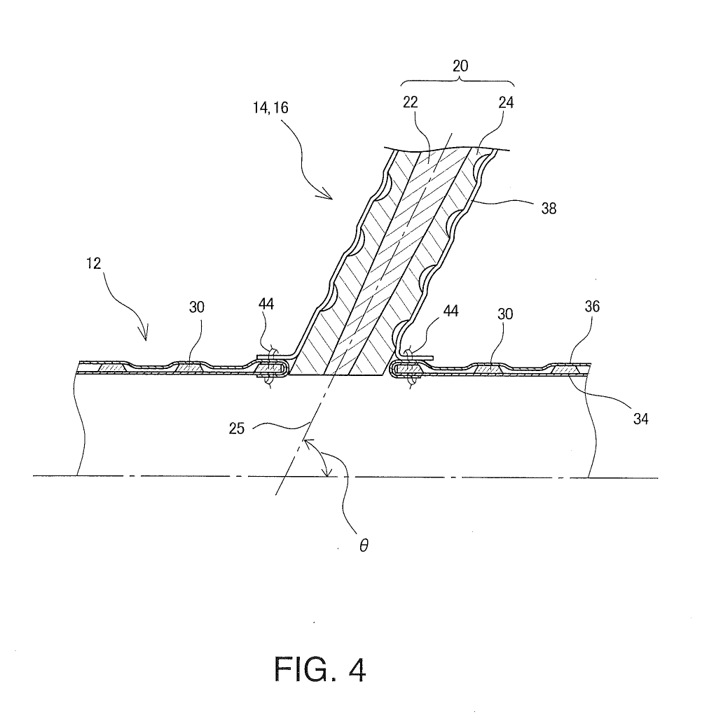

On a ring portion 40 having a ring shape and defining the opening 32 of the reinforcement member 30, suture holes 42 are formed in the circumferential direction. A suturing thread 44 is passed through the suture holes 42, to suture the inner wall 34 and the outer wall 36 which are blood vessel walls of the trunk portion 12 and the blood vessel wall 38 of the branch portion. The blood vessel wall 38 of the branch portion is sutured at the inside of the blood vessel wall of the trunk portion. As described above, because the inner wall 34 entangles the outer side surface and the outer wall 36 entangles the inner side surface, the blood vessel walls are sutured at the outer side and the inner side of the ring portion 40, respectively. FIG. 4 shows an example configuration where the blood vessel wall 38 of the branch portion is sutured at the outer side of the blood vessel wall of the trunk portion.

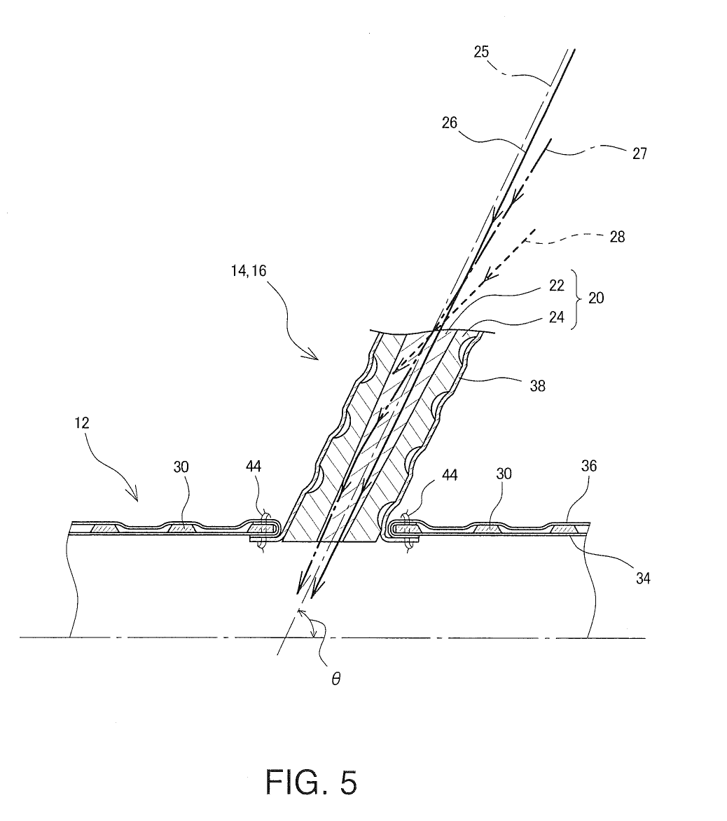

As shown in FIG. 5 with a solid line 26, a needle is inserted from above toward the bottom left direction, that is, along the axis 25 of the branch portions 14 and 16 from the branch portions 14 and 16 toward the trunk portion 12. The center portion 22 has a hardness of a degree to allow the needle to be inserted. As shown in FIG. 5 with a broken line 28, when the needle is inserted in an inclined manner with respect to the axis 25, the tip of the needle contacts the peripheral portion 24. Because the peripheral portion 24 is hard, the needle is not further inserted or the user feels the resistance and stops insertion of the needle. In addition, when the needle is inserted with a shallow angle with respect to the inner wall surface of the peripheral portion 24 as shown in FIG. 5 with a dot-and-chain line 27, the tip of the needle contacts the inner wall surface of the peripheral portion 24 and then moves toward the trunk portion 12 along the inner wall surface of the peripheral portion 24. Thus, the final position of the needle tip is not significantly deviated. In particular, because the inner wall surface of the peripheral portion 24 has a tapered shape, the area of the region where the needle can be inserted at an end in which the needle is inserted (end outside of the body) is widened, and at the same time, the region where the needle protrudes to the blood vessel side can be limited to a narrow region.

In addition, when the needle is inserted, a force to push the branch portions 14 and 16 toward the deeper position is added, and acts in the branching portion to squash the tubular shape of the trunk portion 12 in the lateral direction. However, in the artificial blood vessel 10, because the branching portion is reinforced by the reinforcement member 30, the lateral force is borne by the reinforcement member 30, and the deformation and closure of the trunk portion 12 are prevented. In other words, the reinforcement member 30 has rigidity to a degree to prevent the closure of the trunk portion 12 when bearing the lateral force. For the reinforcement member 30, the material, thickness, aperture of the mesh structure, etc., are suitably changed according to a presumed value of the force acting in the lateral direction. When the needle is pulled out of the access port 20, the hole opened by the needle is closed due to the elasticity of the center portion 22, and the sealed state is recovered.

A cuff which is flocked with a material having a biocompatibility such as SHAp (highly-dispersible nano-particles of sintered hydroxyapatite) may be placed around the branch portions 14 and 16. With the firm tissue-bonding of the cuff and the subcutaneous fibroblast, the infection risk at the boundary between the branch portion and the skin can be reduced.

The access port 20 is not limited to the two-layer structure of a soft center portion and a hard peripheral portion, and may alternatively have a structure where the hardness is increased from the center toward the outer side in multiple stages or continuously.

The above-described artificial blood vessel 10 corresponds to the treatment where the blood is temporarily extracted to the outside of the body and then returned into the body after a predetermined process. A case where a medical agent is injected into the blood vessel can be handled by providing one access port. That is, the artificial blood vessel would be an artificial blood vessel having a medical agent injection access port which uses the left half including the branch portion 14 of FIG. 1. In addition, for the artificial blood vessel of a simple tube and having no branch portion, by placing the tubular reinforcement member, it is possible to inhibit deformation of the blood vessel even when the artificial blood vessel is applied to an arm section or a leg section where a lateral force tends to be applied.

In the present embodiment, a case is exemplified where the trunk portion 12 and the branch portions 14 and 16 are connected by suturing, but the present invention is not limited to such a configuration, and alternatively, for example, the trunk portion and the branch portion may be connected through adhesion. In addition, a case is exemplified in which a two-layer structure is employed for the blood vessel wall on the portion where the reinforcement member is placed, but the present invention is not limited to such a configuration, and alternatively, for example, the entire artificial blood vessel may be formed in the two-layer structure.

Furthermore, in the present embodiment, a case is exemplified in which the recess-projection portion 28 is formed on a part of the outer circumferential surface of the access port, but the present invention is not limited to such a configuration, and alternatively, the recess-projection portion 28 may be formed in a wider range, for example, over the entirety of the outer circumferential surface.

In addition, in the present embodiment, an example configuration is described in which the access port 20 is formed in an approximate circular tube shape, but the present invention is not limited to such a configuration, and for example, the access port 20 may be formed in a shape where the outer diameter is increased toward the surface of the body so that the access is facilitated.

The present invention is not limited to the embodiment described above, and includes all changes and modifications which do not depart from the scope and principle of the present invention defined in the claims.

### EXPLANATION OF REFERENCE NUMERALS

10 ARTIFICIAL BLOOD VESSEL; 12 TRUNK PORTION; 14, 16 ACCESS BRANCH PORTION; 18 SHUNT BRANCH PORTION; 20 ACCESS PORT; 22 CENTER PORTION; 24 PERIPHERAL PORTION; 30 REINFORCEMENT MEMBER

1. An artificial blood vessel, comprising:
a trunk portion which forms a part of the blood vessel; and
a branch portion branched from the trunk portion and in which an access port, into which a needle for extracting a fluid from the body or introducing the fluid into the body is inserted, is placed, wherein
the trunk portion comprises a tubular reinforcement member integrated with a blood vessel wall of the artificial blood vessel, placed at a position where the branch portion branches and an adjacent portion thereto, and having rigidity to prevent closure of the artificial blood vessel when a lateral force is applied, wherein the blood vessel wall of a portion where the reinforcement member is placed has two layers including an inner wall and an outer wall, the reinforcement member is sandwiched between the inner wall and the outer wall, and the reinforcement member is not exposed to the outside, and
at an edge of an opening of the reinforcement member, the blood vessel walls of the trunk portion and the branch portion are folded and connected.

2. (canceled)

3. (canceled)

4. (canceled)

5. The artificial blood vessel according to claim 1, wherein
a tubular wall of the reinforcement member has a mesh shape.

6. (canceled)

7. (canceled)

8. The artificial blood vessel according to claim 1, wherein
an opening is formed in the reinforcement member at a position where the branch portion branches, and the trunk portion and the branch portion are connected at a position of the opening.

9. The artificial blood vessel according to claim 5, wherein
an opening is formed in the reinforcement member at a position where the branch portion branches, and the trunk portion and the branch portion are connected at a position of the opening.

10. (canceled)

11. The artificial blood vessel according to claim 8, wherein
the trunk portion and the branch portion are connected by suturing at the position of the opening.

12. The artificial blood vessel according to claim 8, wherein
a plurality of suture holes are arranged along a circumferential direction in a ring-shaped portion defining the opening, and the trunk portion and the branch portion are sutured by passing a suturing thread through the suture holes.

13. The artificial blood vessel according to claim 11, wherein
a plurality of suture holes are arranged along a circumferential direction in a ring-shaped portion defining the opening, and the trunk portion and the branch portion are sutured by passing a suturing thread through the suture holes.
